(19) United States
(12) Patent Application Publication
(10) Pub. No.: US 2010/0106017 A1
(43) Pub. Date: Apr. 29, 2010

(54) Ultrasound System Capable Of Displaying Plurality Of Divided Screens

(75) Inventors: Soo Hwan Shin, Seoul (KR); Sung In Park, Hanam-Si (KR); Su Myeong Lee, Anyang-si (KR)

Correspondence Address: JONES DAY, 222 EAST 41ST ST, NEW YORK, NY, 10017, US

(73) Assignee: Medison Co., Ltd.

(21) Appl. No.: 12/607,759

(22) Filed: Oct. 28, 2009

(30) Foreign Application Priority Data
Oct. 29, 2008 (KR) 10-2008-0106520

(51) Int. Cl. A61B 8/00; G06K 9/00

(52) U.S. Cl. 600/437; 382/131

(57) ABSTRACT

Disclosed herein is an ultrasound system capable of displaying a plurality of divided screens. The ultrasound system includes an ultrasound diagnosis unit transmitting an ultrasound signal to a patient body and receiving the ultrasound signal reflected from the patient body to generate a reception signal, a data generator generating ultrasound image data based on the reception signal, a memory storing the ultrasound image data, and an image processor dividing a screen of a display unit into a plurality of divided screens according to set-up of the display unit input from a user input unit and displaying an ultrasound image on the respective divided screens.

### CROSS-REFERENCE TO RELATED APPLICATIONS

This application claims the benefit of Korean Patent Application No. 10-2008-0106520, filed Oct. 29, 2008, the entire contents of which are incorporated by reference herein.

### BACKGROUND OF THE INVENTION

1. Field of the Invention

The present invention relates to an ultrasound system and, more particularly, to an ultrasound system that allows a screen of a display unit supporting a widescreen mode to be divided into a plurality of screens according to set-up of the display unit input from a user input unit, such that an ultrasound image can be displayed on the plurality of divided screens.

2. Description of the Related Art

An ultrasound system is a frequently used diagnostic system. In particular, the ultrasound system has been widely applied to medical fields due to its non-invasive and non-destructive characteristics with respect to a patient body. In recent years, high performance ultrasound systems have been used to generate two-dimensional or three dimensional images of the internal organs of the patient body.

Generally, a probe of the ultrasound system is provided with a transducer for transmitting and receiving ultrasound signals in a wide wavelength band. The transducer generates and transmits ultrasound signals to a patient body in response to an electric stimulus. Then, ultrasound echo-signals reflected from the patient body are transmitted to the transducer, where the ultrasound echo-signals are converted into electrical signals. The electrical signals are subjected to amplification and signal processing to generate ultrasound image data.

On the other hand, the ultrasound system provides a function of reading out the ultrasound image data from an internal or external storage inside or outside the ultrasound system to provide an ultrasound image based on the ultrasound image data read out therefrom, and a function of obtaining ultrasound image data based on signals received in real time through the probe to provide ultrasound images based on the obtained ultrasound image data.

Here, it should be noted that the above description is not given for illustration of a conventional technique to which the present invention pertains, but for understanding of the background of the present invention.

A conventional ultrasound system is configured to display an ultrasound image only within some regions on a screen of a display unit. With this configuration, when using a dual mode (for dividing an image region into upper and lower or right and left regions) or a quad mode (for dividing the image region into a 2×2 structure) to display the ultrasound image, right and left edges of the ultrasound image can be cut or the size of the ultrasound image can be reduced, thereby making it difficult and inconvenient for a user to efficiently recognize the ultrasound image during ultrasound diagnosis with the system. Therefore, there is a need for an improved ultrasound system that overcomes such a problem.

### SUMMARY OF THE INVENTION

The present invention is conceived to solve the problem of the conventional technique as described above, and an aspect of the present invention is to provide an ultrasound system that allows a screen of a display unit supporting a widescreen mode to be divided into a plurality of screens arranged in a desired layout according to set-up of the display unit input from a user input unit to display an ultrasound image on the plurality of divided screens without cropping or size-reducing the ultrasound image, so that a user can recognize the ultrasound image efficiently to carry out more accurate ultrasound diagnosis.

According to an aspect of the present invention, an ultrasound system capable of displaying a plurality of divided screens includes an ultrasound diagnosis unit transmitting an ultrasound signal to a patient body and receiving the ultrasound signal reflected from the patient body to generate a reception signal; a data generator generating ultrasound image data based on the reception signal; a memory storing the ultrasound image data; and an image processor dividing a screen of a display unit into a plurality of divided screens according to set-up of the display unit input from a user input unit and displaying an ultrasound image on the respective divided screens.

The display unit may include a display supporting a widescreen mode.

The image processor may form the ultrasound image based on a plurality of data frames input from at least one of the data generator and the memory.

The ultrasound system may further include an external image storage storing external image data obtained from an external image apparatus.

The image processor may form the ultrasound image based on a plurality of data frames input from at least one of the data generator, the memory, and the external image storage.

The image processor may set at least one box for a region of interest in the ultrasound image and perform image processing with respect to the ultrasound image within the box for the region of interest.

The image processing with respect to the ultrasound image may include at least one of outputting, storing, and transmitting the ultrasound image.

The ultrasound system according to one embodiment of the present invention allows the screen of the display unit supporting a widescreen mode to be divided into a plurality of screens arranged in a desired layout according to set-up of the display unit input from the user input unit to display an ultrasound image on the plurality of divided screens without cropping or size-reducing the ultrasound image, so that a user can obtain the ultrasound image efficiently to carry out more accurate ultrasound diagnosis.

Further, the ultrasound system according to one embodiment of the present invention allows a user to set up at least one box for a region of interest in the divided ultrasound image to perform image processing, so that the user can perform image processing more easily with respect to the specific region of the ultrasound image.

### BRIEF DESCRIPTION OF THE DRAWINGS

The above and other aspects, features and advantages of the present invention will become apparent from the following description of a preferred embodiment given in conjunction with the accompanying drawings, in which:

FIG. 1 is a block diagram of an ultrasound system according to an exemplary embodiment of the present invention;

FIG. 2 illustrates a display unit of the ultrasound system according to an exemplary embodiment of the present invention;

FIGS. 3 and 4 illustrate examples of an ultrasound image displayed on an ultrasound image region by a conventional ultrasound system;

FIGS. 5 to 11 illustrate examples of an ultrasound image displayed on an ultrasound image region by an ultrasound system according to an exemplary embodiment of the present invention; and

FIG. 12 illustrates a region of interest in an ultrasound image set by an ultrasound system according to an exemplary embodiment of the present invention.

### DETAILED DESCRIPTION OF THE EMBODIMENT

Exemplary embodiments of the present invention will now be described in detail with reference to the accompanying drawings hereinafter. It should be noted that the drawings are not to precise scale and may be exaggerated in thickness of lines or size of components for descriptive convenience and clarity only. Furthermore, terms used herein are defined by taking functions of the present invention into account and can be changed according to the custom or intention of users or operators. Therefore, definition of the terms should be made according to overall disclosures set forth herein.

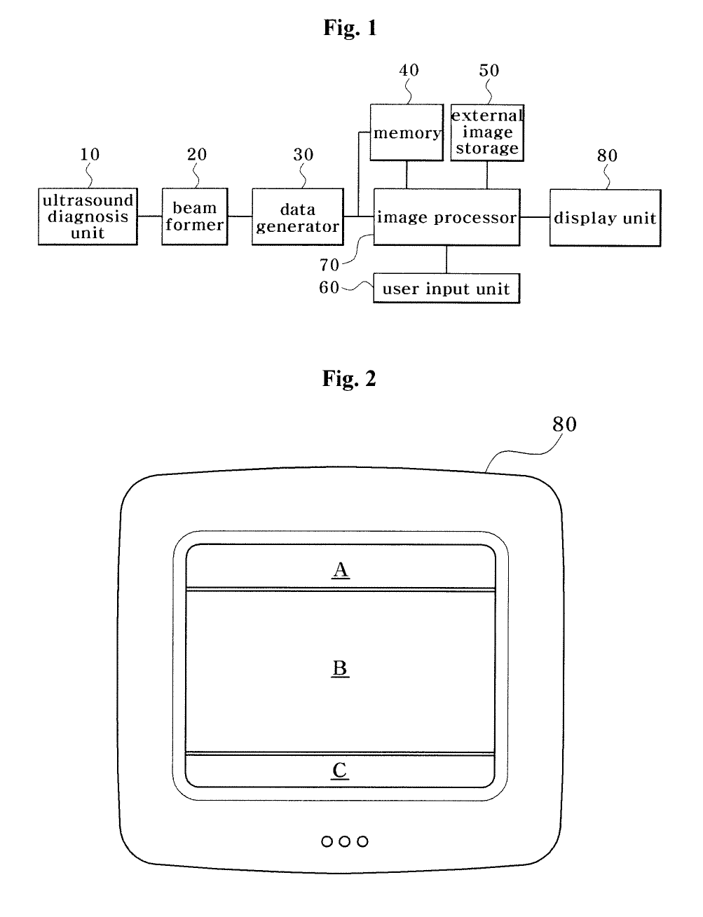

FIG. 1 is a block diagram of an ultrasound system according to an exemplary embodiment of the present invention.

As shown in FIG. 1, the ultrasound system according to the embodiment of the invention includes: an ultrasound diagnosis unit 10 transmitting an ultrasound signal to a patient body and receiving the ultrasound signal reflected from the patient body to generate a reception signal; a beam former 20; a data generator 30 generating ultrasound image data based on the reception signal; a memory 40 storing the ultrasound image data; a user input unit 60; an image processor 70 dividing a screen of a display unit 80 into a plurality of divided screens according to set-up of the display unit input from the user input unit 60 and displaying an ultrasound image on the respective divided screens; and the display unit 80.

Operation of the ultrasound system capable of displaying a plurality of divided screens according to the embodiment of the invention will now be described in detail.

The ultrasound diagnosis unit 10 transmits an ultrasound signal to a patient body and receives the ultrasound signal reflected from the patient body to generate a reception signal. The data generator 30 generates ultrasound image data based on the reception signal.

When viewing a plurality of ultrasound images during ultrasound diagnosis with the system, a user manipulates the user input unit 60 to set up the display unit in a desired layout.

The image processor 70 divides a screen of the display unit 80 into a plurality of divided screens according to set-up of the display unit 80 input from the user input unit 60, and displays an ultrasound image on the respective divided screens based on a plurality of data frames input from at least one of the data generator 30 and the memory 40.

To provide an external ultrasound image among the plurality of ultrasound images, the ultrasound system may further include an external image storage 50 which stores external image data obtained from an external image apparatus. Then, the image processor 70 forms a plurality of divided screens and displays the ultrasound image on the respective divided screens based on a plurality of data frames input from at least one of the data generator 30, the memory 40, and the external image storage 50.

The display unit 80 may include a display supporting a widescreen mode. For example, a widescreen monitor may be used for the display unit 80.

FIG. 2 illustrates a display unit of the ultrasound system according to an exemplary embodiment of the present invention.

As shown in FIG. 2, a display unit 80 of the ultrasound system according to an exemplary embodiment of the invention includes a title region A where logos, patient information, hospital names and diagnosis subjects, frame rates and depth, probe information, date and time, etc. are displayed, an image region B where ultrasound images, image information, annotation, measurement information, etc. are displayed, and a feedback region C where a current selection mode, a state of trackball function, image freeze, etc. are displayed.

The ultrasound system of the present invention displays an ultrasound image on a plurality of divided screens, and embodiments of the present invention will be now described with reference to the image region B where the ultrasound image is displayed.

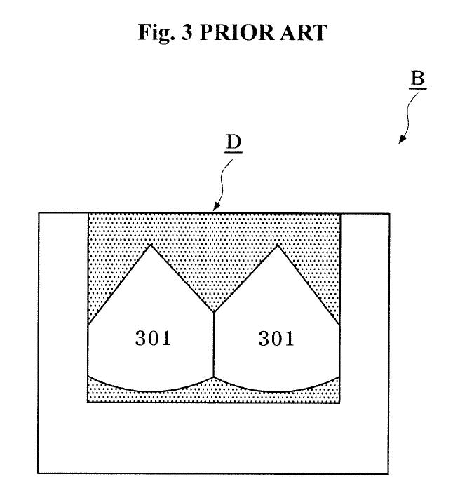

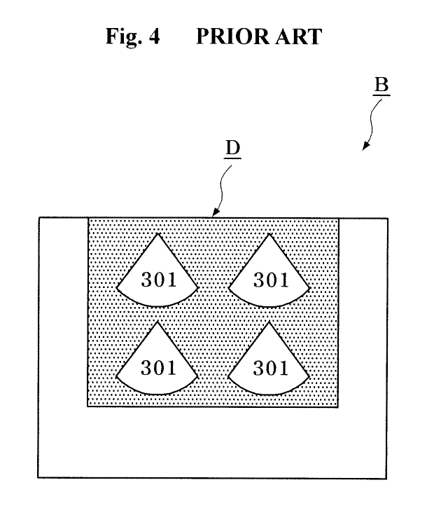

FIGS. 3 and 4 show examples of an ultrasound image displayed on an image region B by a conventional ultrasound system.

FIG. 3 illustrates one example of a screen of the ultrasound system which displays an ultrasound image in a dual mode of the conventional ultrasound system. In the dual mode, a single ultrasound image region D is divided into right and left regions. In this case, the right and left edges of an ultrasound image 301 can be cut, as shown in FIG. 3.

FIG. 4 illustrates one example of the screen of the ultrasound system which displays an ultrasound image in a quad mode of the conventional ultrasound system. In the quad mode of the screen, the ultrasound image region D is divided into four regions, that is, upper, lower, right, and left regions. In this case, the size of the ultrasound image 301 can be reduced, as shown in FIG. 4.

As such, in the conventional ultrasound system, the ultrasound image region D of the display generally has a size of 1024×768, thereby providing inconvenience to a user when displaying a plurality of ultrasound images in the dual mode or the quad mode.

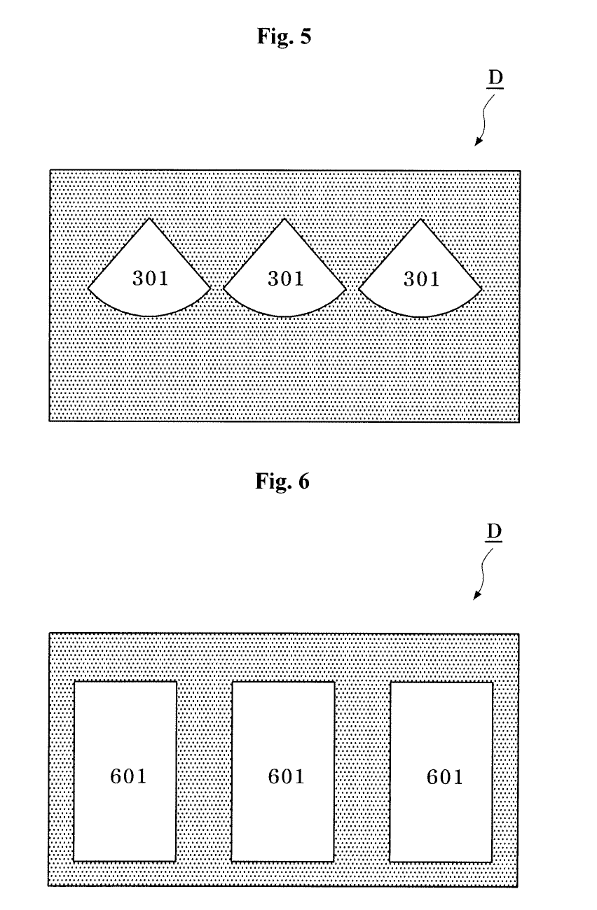

FIGS. 5 to 11 show examples of an ultrasound image region D displayed by an ultrasound system according to one exemplary embodiment of the present invention.

In a widescreen monitor, the ultrasound image region D may have a size of 1680×1050.

FIGS. 5 and 6 show the ultrasound image region D which is transversely divided into three regions on the widescreen monitor of the ultrasound system according to the embodiment of the invention.

Referring to FIG. 5, each of three divided ultrasound images 301 has a length of 560 pixels, which is greater than a length of 512 pixels in the dual mode of the conventional ultrasound system, thereby preventing cropping or size reduction of the ultrasound image.

Referring to FIG. 6, each of three divided ultrasound images has a greater height than length, so that a deep ultrasound image 601 can be more efficiently observed.

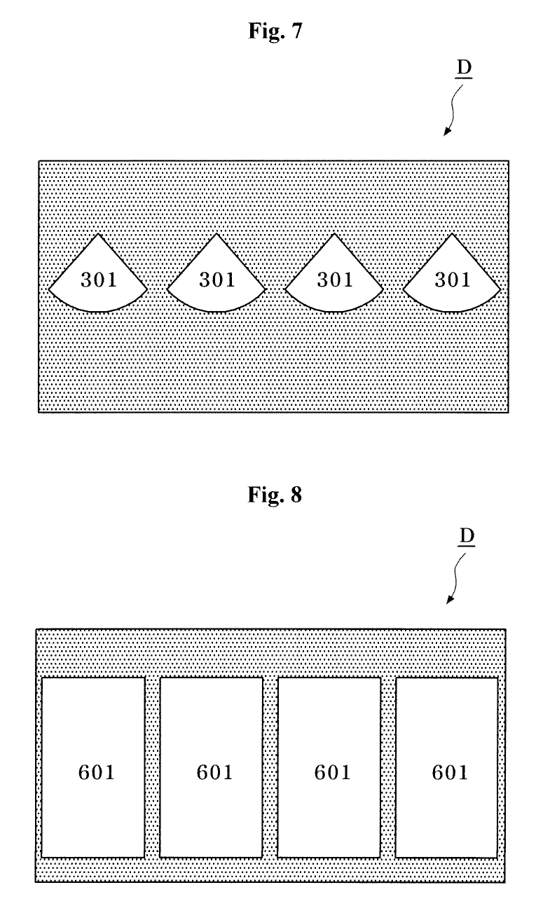

FIGS. 7 and 8 show the ultrasound image region D which is transversely divided into four regions on the widescreen monitor of the ultrasound system according to the embodiment of the invention.

Referring to FIG. 8, each of four divided ultrasound images 301 has a length of 420 pixels, which is greater than the size of the ultrasound image in the quad mode of the conventional ultrasound system, thereby preventing cropping or size reduction of the ultrasound image.

Referring to FIG. 8, each of four divided ultrasound images has a greater height than length, so that a deep ultrasound image 601 can be more efficiently observed.

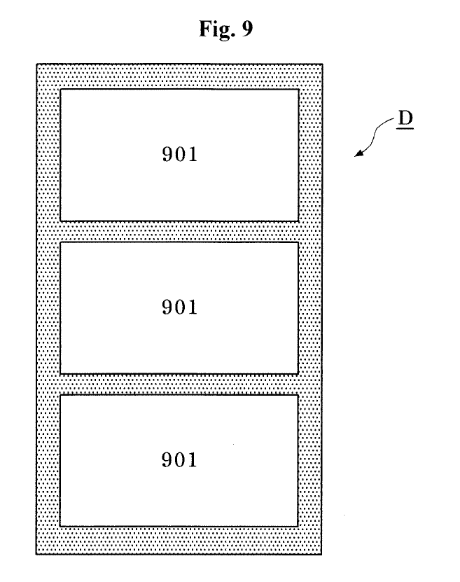

FIGS. 9 to 11 show the ultrasound image region D which is longitudinally divided on the widescreen monitor of the ultrasound system according to the embodiment of the invention, in which the widescreen monitor is rotated at 90 degrees.

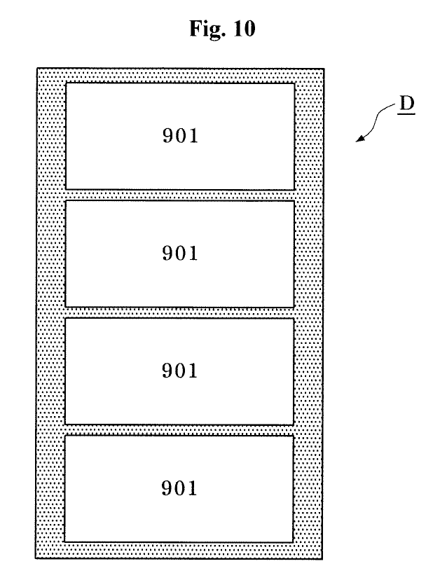

Referring to FIGS. 9 and 10, with the widescreen monitor of the ultrasound system rotated at 90 degrees, the ultrasound image region D is longitudinally divided into three or four regions. In this case, each of divided ultrasound images has a greater length than height, so that an ultrasound image 901 having an increased length, such as a panoramic image, can be more efficiently observed.

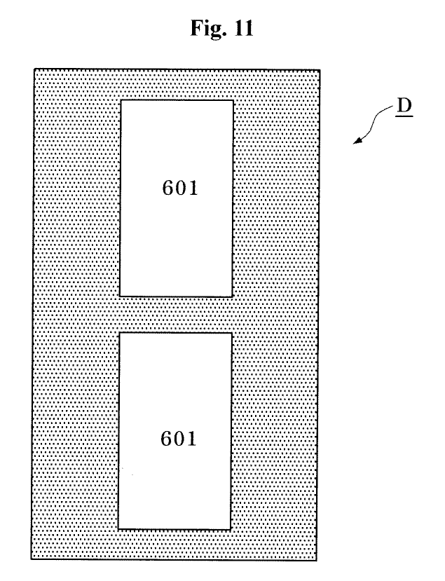

Referring to FIG. 11, a user can observe deep ultrasound images 601 by dividing an ultrasound image into ultrasound images 601 after rotating the widescreen monitor of the ultrasound system at 90 degrees, as needed.

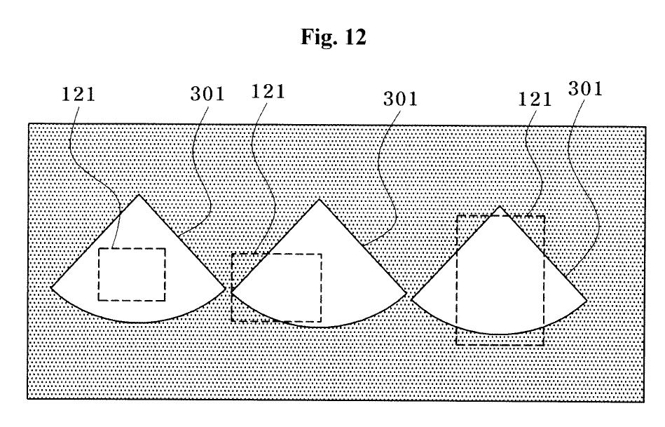

FIG. 12 illustrates a region of interest in an ultrasound image set by an ultrasound system according to an exemplary embodiment of the present invention.

Referring to FIG. 12, a user sets a box 122 for a region of interest in an ultrasound image 301 to perform image processing with respect to a specific region in the ultrasound image. Then, the image processor performs image processing with respect to the region of interest selected by the user with ultrasound image data. For example, the image processing with respect to the region of interest selected by the user may include at least one of outputting, storing, and transmitting the ultrasound image.

As such, the ultrasound system according to one embodiment of the present invention allows a screen of a display unit supporting a widescreen mode to be divided into a plurality of screens arranged in a desired layout according to set-up of the display unit input from a user input unit to display an ultrasound image on the plurality of divided screens without cropping or size-reducing the ultrasound image, so that a user can obtain to the ultrasound image efficiently to carry out more accurate ultrasound diagnosis.

Further, the ultrasound system according to another embodiment of the present invention allows a user to set up at least one region of interest in the divided ultrasound image to perform image processing, so that the user can perform image processing more easily with respect to the specific region of the ultrasound image.

Although the present invention has been described with reference to the embodiments, it will be apparent to those skilled in the art that the embodiments are given by way of illustration only, and that various modifications and equivalent embodiments can be made without departing from the spirit and scope of the present invention. Accordingly, the scope of the present invention should be limited only by the accompanying claims.

What is claimed is:

1. An ultrasound system displaying a plurality of divided screens, comprising:
an ultrasound diagnosis unit transmitting an ultrasound signal to a patient body and receiving the ultrasound signal reflected from the patient body to generate a reception signal;
a data generator generating ultrasound image data based on the reception signal;
a memory storing the ultrasound image data; and
an image processor dividing a screen of a display unit into a plurality of divided screens according to set-up of the display unit input from a user input unit and displaying an ultrasound image on the respective divided screens.

2. The ultrasound system according to claim 1, wherein the display unit comprises a display supporting a widescreen mode.

3. The ultrasound system according to claim 1, wherein the image processor forms the ultrasound image based on a plurality of data frames input from at least one of the data generator and the memory.

4. The ultrasound system according to claim 1, further comprising:
an external image storage storing external image data obtained from an external image apparatus.

5. The ultrasound system according to claim 4, wherein the image processor forms the ultrasound image based on a plurality of data frames input from at least one of the data generator, the memory, and the external image storage.

6. The ultrasound system according to claim 1, wherein the image processor sets at least one box for a region of interest in the ultrasound image and performs image processing with respect to the ultrasound image within the box for the region of interest.

7. The ultrasound system according to claim 6, wherein the image processing with respect to the ultrasound image comprises at least one of outputting, storing, and transmitting the ultrasound image.
